(19) United States
(12) Patent Application Publication
(10) Pub. No.: US 2020/0405895 A1
(43) Pub. Date: Dec. 31, 2020

(54) DEVICE FOR DISINFECTING PIPELINES, CONTAINERS AND STRUCTURES

(71) Applicant: Manfred MOCK, Pöllau (AT)

(72) Inventor: Manfred MOCK, Pöllau (AT)

(21) Appl. No.: 16/976,555

(22) PCT Filed: Mar. 13, 2019
(86) PCT No.: PCT/AT2019/060082

§ 371 Date: Aug. 28, 2020

(30) Foreign Application Priority Data
Mar. 22, 2018 (AT) A50240/2018
Dec. 13, 2018 (AT) A51116/2018

(51) Int. Cl. A61L 2/10; A61L 2/26; A61L 2/22

(52) U.S. Cl. CPC A61L 2/10; A61L 2/26; A61L 2202/23; A61L 2202/15; A61L 2202/11; A61L 2/22

(57) ABSTRACT

A device for disinfecting pipelines, containers and structures using disinfectants and/or electromagnetic radiation. Water is drained from pipes, containers and structures through which water flows. They are then preliminarily cleaned, and disinfectants are dispensed as a mist via spray heads provided on a spray chain that is moved through the pipe, container or structure. The spray chain can have CO₂ nozzles and associated CO₂ supply equipment. Alternatively, or in addition, the drained pipes, containers and structures are disinfected using electromagnetic radiation emitted by means of radiation sources.

The invention relates to a device for the disinfection of pipes, vessels, and structures according to the preamble of claim 1.

In (drinking) water-carrying pipes, containers or structures, regardless of their cross-section, strong biofilms form over time; these form on the inner pipe wall and serve as the basis of life for microorganisms. As long as these biofilms are free from harmful germs and pathogens, this is not a problem.

However, pathogenic germs (bacteria and/or viruses such as Escherichia coli, Enterococci, Legionella or Pseudomonas aeruginosa), along with fungi and their spores (and, if exposed to light, algae), can colonize within these biofilms, and can flow out with the water flowing through the pipe. These microorganisms can then reach individual consumers (e.g. through drinking water supply systems in the individual households) and cause a health hazard for humans and animals.

One must therefore clean, sample and, if required, disinfect drinking water supply systems.

### Disinfection:

Disinfection places a system in a state (milieu) in which microorganisms are killed or damaged in such a way that growth or reproduction is no longer able to reoccur. Furthermore, disinfection also means that viruses are inactivated. This state is generally time-limited and may have to be re-established again and again.

The current commonly used methods of disinfecting (drinking) water-flow pipes, containers or structures are based on the use of aqueous disinfectant solutions or gases.

In this process, aqueous disinfectant solutions are introduced into, or run through, the components to be disinfected using the continuous flow process or static method.

For drinking water supply systems, measures are to be applied according to the specifications of ÖVWG RL W 55 or DVGW W 291, using the aforementioned disinfectant.

The following disinfection techniques are currently used (without explanation of pre-cleaning and rinsing):
In the continuous-flow process, a disinfectant, typically chlorine or chlorine solution, is usually added to the water flowing in a pipe in different concentrations and at different times.
In the static method, the disinfectant solution used for disinfection (e.g. more chlorinated water) is left in the pipelines for a longer period of time (approx. 24-48 hours) and then drained off.

The disadvantages of the aforementioned techniques are that the disinfectant solutions to be disposed of are produced in large quantities, and that e.g. the chlorinated water has to be removed from the pipes and containers.

### Problem

Despite the disinfection method that has been described, the persistence of very resistant germs, such as Pseudomonas aeruginosa, in the biofilm is a problem. Once this germ has manifested itself in the biofilm, chemical disinfection—as mentioned above—does not offer any possibility of longterm removal of this pathogen from the tube or biofilm. When chlorine is used, it protects itself with a wax-like layer and “sits” out the hazardous situation, so to speak.

The aim and object of the invention is to provide a device which is capable of coping with such resistant germs.

According to the invention, these objectives are achieved by a device which has the features specified in the characterizing part of claim 1. In other words, a spray chain moves through the emptied and possibly pre-cleaned pipeline, the container or the structure, with at least one nozzle for dispensing and atomizing the disinfectant, and/or at least one source of electromagnetic radiation, particularly a UVC source. The spray chain (“spray” due to the spraying of a mist and/or electromagnetic radiation; the latter is more useful the “harder” it is) is, as explained in more detail below, constructed in such a way that the nozzle and/or source is separated from the floor and the sidewalls of the pipe, container or structure.

Even if the light source is dispensed with, due to the atomization, the effectiveness of the disinfectant is significantly increased and the amount of the agent that needs to be introduced is significantly reduced.

The inventive device has a spray chain which is introduced into the pre-cleaned or unpurified pipeline, the container or the building and activated there, thus spraying the disinfectant in a nebulized form and is controlled by a so-called operating drum, which is not introduced into the structure. The spray chain can be pulled by a traction device (chain, rope, etc.) previously inserted into the pipeline, the container or the structure; it can be attached to a (remotely controlled) vehicle; or it can be moved through the area to be cleaned in some other way.

The spray and mist nozzles can also be used to introduce alcohol into the building, which destroys the entire biofilm and prevents the pathogenic germs from multiplying and spreading. The high-pressure spraying of alcohol can also bring about a mechanical detachment of the biofilm. Mechanical shearing is involved, so to speak.

The provision of corresponding light sources, their energy supply via power lines and the activation by means of switches can also be directly understood without further explanation, and in most cases several sources of this type (pointing in different directions) are provided.

UV treatment is the state of the art in disinfection for drinking water treatment. However, drinking water is conducted in a run-through process through so-called UVC disinfection systems and then fed into the drinking water network. These systems can also be installed directly in the water pipes themselves, or the UV system can be installed within an existing water supply system immediately after water extraction and eventual treatment, before the distribution network and all available storage tanks. Their use for cleaning empty pipes or structures is not known.

Given the present state of the art, by filling of the system, the container or the pipe with clean water after cleaning and usual rinsing with pure water, which is drained off and not put to further use, the natural biofilm settlement process may return over time.

By using the inventive technology, all of the disadvantages mentioned above can be avoided and even Pseudomonas aeruginosa can be effectively combatted, thus achieving long-term sterility in drinking water-filled pipes and containers or structures that are filled with drinking water, and meeting the legal requirements.

All known disinfectants, liquid or gaseous, can be used with this invention; examples are:

Halogen-containing chemicals such as chlorine, chlorine bleach (sodium hypochlorite solution), chlorine lime (calcium hypochlorite), chlorine dioxide; oxygen-containing chemicals such as hydrogen peroxide, potassium permanganate, ozone, peracetic acid; nitrogen-containing chemicals such as quaternary ammonium compounds, benzalkonium chloride, cetylalkonium chloride; alcohols, such as isopropanol.

To combat so-called invertebrates, CO₂ gas can be independently introduced into the water pipe or the structure via the spray chain. These tiny organisms, which cling to the biofilm, are anesthetized in the process, meaning that they can be removed from the biofilm and rinsed out with the detergent, or with rinsing water. Appropriate nozzles can be fitted on the spray chain in addition to the above-mentioned. The CO₂ is supplied through one or more hoses, and the delivery is controlled by remote-controlled solenoid valves.

Light in the particular wavelength range between 200-300 nanometers (nm) with a pronounced maximum of approx. 265 nm is suitable for the disinfection of drinking water. The optimal wavelength can vary slightly depending on the microorganism and may be taken into account. One advantage of such UV disinfection process over conventional methods (chemical disinfection) is the fact that bacteria and viruses are unable to build up resistance to ultraviolet radiation; when combined, the effect is markedly greater than when UV and chemical disinfection are used separately.

Ultraviolet light-emitting diodes (UV LEDs) have already been proven to be able to disinfect water. Irradiation with UV light destroys the genetic makeup of bacteria, viruses and spores (i.e. of microbes in general), thereby preventing the multiplication of organisms. In special cases, even shorter-wave beams can be used to an advantage, even if their emission must be subject to special security measures.

Even when faced with all kinds of stubborn germs that colonize on the walls of water-bearing structures, the innovative method (particularly the combination variant) has been proven to make compliance with legal hygiene requirements possible.

The invention is described in more detail below with with the help of the diagram which shows, in purely diagrammatic form:

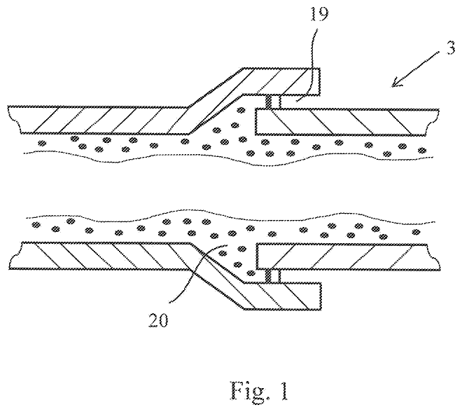

In FIG. 1, a pipe union with impurities;

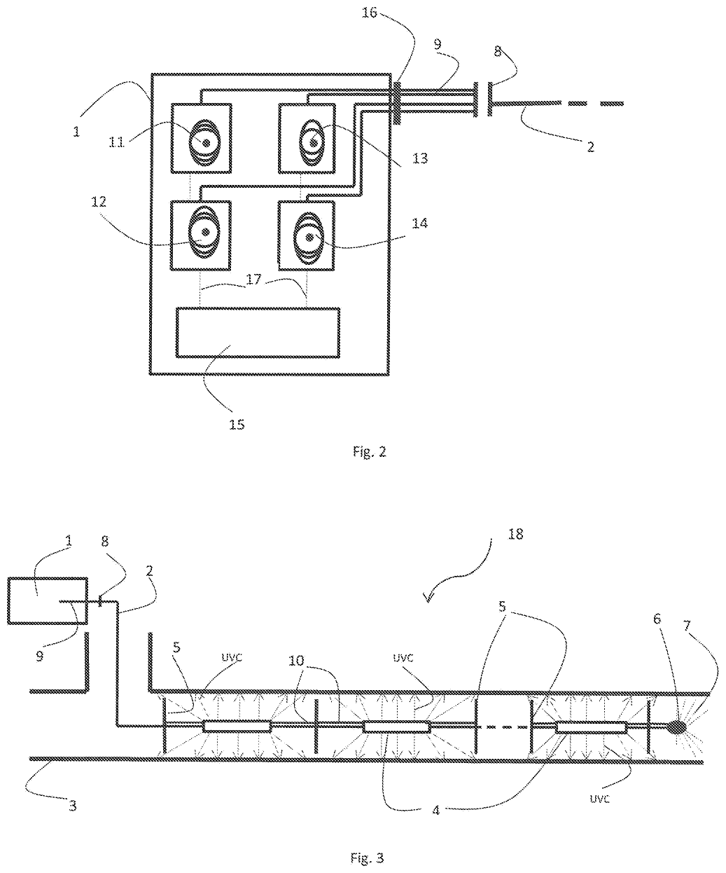

In FIG. 2, a so-called operating drum;

In FIG. 3, a usable spray chain in lateral view; and

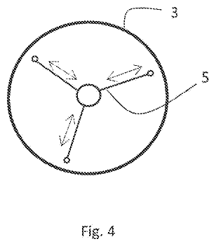

In FIG. 4, the same in axial view.

When using the method, a spray chain is preferably equipped with at least one, and preferably multiple, spray heads for dispensing the disinfectant (referred to in brief as “agent”) by means of a so-called operating drum, as is shown in the diagram in FIG. 2. By dispensing the agent in the form of a fine spray mist, its effectiveness increases, presumably due to the enlargement of the surface, its strong curvature, and the intensive contact with the oxygen in the ambient air.

With the invention, UVC or UV LED lamps, as shown in the diagram in FIG. 3, can be introduced into the pipeline system which is as empty and pre-cleaned as possible; then the lamps are activated and the pipe is irradiated from within while observing certain reaction times. The combination increases the effectiveness of the agent, presumably due to the further increased energy input.

Extra pipes and nozzles can be independently provided for the delivery of CO₂.

For operating drum 1: It is the central supply and control unit for the spray chain. The actual control unit 15 controls the unwinding and winding units of 11, 12, 13 and 14 over the control lines 17 and winds or unwinds the connecting hoses and cables depending on the length used. The connecting hoses for CO₂, disinfectants, electr. power, data cables and camera cables are combined in a coupling 8 and via an outlet roller 16 derived from the operating drum 1. The spray chain is coupled to the connecting hoses or connecting cables via the coupling 8. CO₂, disinfectant, etc. and electrical current are externally fed into the corresponding connection points on the unwinding or winding units 11, 12, 13 and 14. A pump is integrated in 12, which pumps disinfectant into the connecting hose 12 for subsequent atomization via a spray head 6. CO₂ is fed into the connection point at 11 under pressure using a gas pressure bottle.

The supply lines and supply cables 9, each with a length of up to 500 m, are wound up on the unwinding or winding units 11, 12, 13 and 14 and function like an electrically operated reel. After the coupling 8, the supply lines 9 are combined into a single—wound—supply line 2. The operating drum 1, can be mounted as a whole unit on its own carrier or trailer, or in a motor vehicle.

The spray chain can be inserted and moved in various ways: a traction device can be inserted first and then the spray chain can be pulled, the spray chain can be pushed under certain circumstances, or a pulling vehicle could be attached to the head of the spray chain.

The operating drum serves as a control system for regulating the spray chain, the speed of advance within the pipe and thus the duration of the irradiation and general process monitoring and process logging.

In piping systems 3, a disinfectant is particularly directed towards parts that are difficult to access. This has a particularly bactericidal effect and can also penetrate into remote corners in atomized or nebulized form. In addition, the spray mist is able to penetrate biofilms 20 and can therefore act vertically. This spraying process can be carried out independently of the other spray chain operations, preferably through the spray nozzles provided on the spray chain. The fluid with the agent can be supplied via a hose which is attached to the spray chain.

If there are additional invertebrates (invertebrates) in the biofilm and these cannot be removed by classic rinsing processes, it is possible to use gaseous carbon dioxide (CO₂). When used for a longer period of time, CO₂ can anesthetize microorganisms, which then drain off the biofilm and can be flushed out of the pipe by subsequent water rinsing.

Both the aqueous disinfectants and the gaseous ones are preferably introduced into the pipe by means of pressure lines which are connected to the spray chain and sprayed appropriately by means of pumps and remote-controlled valves. In special cases, the fluid can also be pumped from a moving storage tank.

For spray chain 18, the following must be carried out: It essentially consists of the supply line 2, the spray heads 6, the spacers 5 and connection adapters 10, and (one or more) radiation sources 4. Depending on the required length, it is preassembled, pushed into the pipe 3, then coupled to the coupling 8 and then fully inserted into the pipe 3.

Depending on the application, the spray chain can either
be mechanically inserted
washed with the water in the pipe,
blown with compressed air,
be pulled into the pipe 3 by means of a cable pull or robot

The spray chain is thus introduced into the pipe 3 with the spacers 5 oriented approximately radially to its “spine”, the length of which is variable and dependent on the diameter of the pipe (generally the fluid conductor) the UVC radiation sources 4, the connecting adapter 10 and spray heads 6. The spray chain is connected to the supply lines of the operating drum 1 by means of a coupling 8. The connection adapters 10 have an integrated cable outlet and are favorable for the stability of the spray chain in the longitudinal direction. The spacers 5 (at least three in the same or adjacent axial position are recommended) ensure the stability of the spray chain in the transverse direction.

The duration of misting and/or the duration and intensity of radiation from the light lamps depends on the general level of pollution, on the strength/thickness of the biofilm and the pipe material, and, according to internal investigations, averages 0.5-4 seconds or 0.1-10 watts/cm². Longer irradiation times and higher intensities are of course also possible. At the end of the treatment, or even during the treatment, the mist can be disposed of or kept in motion (to and fro) by fans on at least one side of the building that is to be cleaned in order to, on the one hand, quickly return to operation after cleaning and, on the other hand, to increase the effect through movement.

The electrical current for the lamps 4, is supplied via the supply line 2, the disinfectant (generally: the agent) for spraying is supplied by means of the spray heads 6, and the CO₂ for gassing is introduced into the pipe.

The operating drum 1 also provides the energy source for supplying the lamps 4 with electr. power. The supply of disinfectant and CO₂ is also ensured via the operating drum 1. The disinfectant and the gas are introduced into the pipe 3 by means of pumps via the supply line 2. The disinfectant atomization and CO₂ gas are delivered via the spray heads 6, which are individually and optionally activated by means of solenoid valves.

The preliminary cleaning—pre-cleaning of the pipes etc.—can be done through rinsing with water or through pigging. If there are any invertebrates, CO₂ is used for fumigation and, if necessary, the pipes are rinsed or pigged again. The next step is disinfection using UVC light and/or the agent. The agent is pumped into the pipe 3 by means of its own pressure line, which is integrated into the supply line 2, and sprayed locally by means of the spray heads 6. Finally, the pipe is rinsed out with water.

The diameter of the pipes into which a spray chain (each adapted to the construction) can be inserted is min. 1 inch (25.4 mm) and max. DN 1500 mm; in the case of a non-circular cross-section, analog values apply which can easily be determined by someone with knowledge of the invention.

The spray nozzles 6 are preferably in consecutive differing circumferential directions in order to quickly and reliably achieve uniform misting of the interior and thus the entire surface of the building (pipe) when the spray heads are approximately in the center of the component/pipe.

Depending on the pipe diameter, there are spacers of different sizes between the individual radiators 4 5, which ensure that the emitters are approximately (+/−10th %) positioned in the center of tube 3 and so that the UVC rays are evenly emitted throughout the pipe 3. The number of UVC radiation sources 4 can be altered depending on the application and depends on the degree of contamination, the required duration (irradiation time) in the pipe 3, the intended working time and the like. If there is a large deviation from the circular cross-section in buildings, an adjustment can be made without any problems so long as the geometry is known.

As FIG. 4 clearly shows, the spray chain 18 essentially has radially protruding spacers 5, which, even when the spray chain is rotated about the longitudinal axis, ensure that it is always kept at a distance from the floor and the side walls.

Depending on the diameter of the pipe 3, the spacers 5 can be mounted with different lengths (for example adjustable in length, indicated with “<- - - - ->” in FIG. 4, or exchangeable).

The spacers 5 generally consist of three arms offset from one another in the circumferential direction at different angles (preferably 120°), which preferably have rollers (only indicated, without reference numerals) mounted on the respective outer end of each arm, which enable easier movement within the pipe 3. Such arms are provided on the chain of the spray chain at a suitable axial distance as required.

The spacers can also be arranged axially and offset in the circumferential direction in a helical pattern. This is easily done by a specialist with knowledge of the invention.

In the axial center of the spacers 5 there are connection couplings at the front and rear, enabling the connection adapter 10 to be coupled here. Connection adapters 10 and spacers 5 each have a free cross-section in the middle for the passage of the supply line 2.

The UVC lamp is coupled to the connection adapter 10 (with seal), giving it the rigidity required to insert the spray chain 18 into the pipe 3 and to move it. In addition to this rigidity, the spray chain has a level of mobility in its “spine” that enables it to follow any curvature of the pipe or structure, especially when it is designed to always be pulled. This can be achieved using ball joints or, more simply, ensuring sufficiently large clearance of the connecting elements from chain link to chain link (vertebrae).

As was mentioned before, it is possible to mount rollers or sliding shoes on the free ends of the spacers 5 for greater ease of movement. They are preferably elastic, at least in the “longitudinal direction”, in order to overcome irregularities more easily through elastic deformation.

The spray chain 18 can be equipped with at least one hose for the disinfectant and/or the CO₂ (carbon dioxide), with an outlet being provided at one point at a minimum. There is preferably a plurality of outlets which can be opened in a targeted manner and pointing in different circumferential directions in order to be able to spray fluid in different directions. The provision of a camera with data transmission to the outside and possibly a source of light in the visible wave range at this point/these points is advantageous for controlling the fluid delivery and for monitoring the success of the cleaning process.

It should also be pointed out that in the description and the claims, descriptions such as “lower region” of a hanger, reactor, filter, structure, or a device or, more generally, an object refers to the lower half and particularly to the lower quarter of the total height; “lowest region” refers to the lowest quarter (or less); while “middle area” refers to the middle third of the total height (width−length). All this information has its common meaning, applied to the intended position of the object under consideration.

In the description and the claims, the terms “front”, “rear”, “top”, “bottom” and so on are used in the usual form and with reference to the object in its usual position of use. This means that with a weapon, the muzzle of the barrel is “in front”, that the bolt or slide is moved “backwards” by the explosion gases, etc. In the case of the spray chain, direction of movement refers to the movement during the cleaning process.

In the description and claims, “essentially” means a deviation of up to +1-10% of the given value, if physically possible, otherwise only in the sensible direction; in the case of degrees (angle and temperature), it means±10°.

All quantities and proportions, particularly those that delimit the invention, insofar as they do not relate to the specific examples, are to be understood to have a ±10% tolerance. For example: 11% means: between 9.9-12.1%. For terms such as: “a solvent”, the word “a” is not to be regarded as a numerical word, but as an indefinite article, unless the context indicates otherwise.

The term: Unless otherwise stated, “combination” or “combinations” refers to all types of combinations, starting from two of the constituents concerned to a large number of such constituents. The term: “containing” also means “consisting of”.

### REFERENCE SYMBOL LIST

1 Operating drum
2 Supply line
3 Pipe (fluid conductor)
4 Radiation source
5 Spacers
6 Spray nozzle
7 Disinfectant spray
8 Coupling
9 Supply lines from the operating drum
10 Connection adapter
11 Wind and unwind unit for the connection hose for gas
12 Wind and unwind unit for the connection hose for disinfectants
13 Wind and unwind unit for the connection hose for the cable for the power supply
14 Wind and unwind unit for the connection hose for the cable for the camera
15 Control unit
16 Outlet roller
17 Control lines
18 Spray chain in total
19 Connection adapter
20 Biofilm

1-8. (canceled)

9. A device for disinfecting pipelines, containers, and structures, each pipeline, container, and structure having a floor and lateral walls, the device comprising:
at least one spray head configured to dispense disinfectant and/or at least one source of electromagnetic radiation, wherein each spray head and/or electromagnetic radiation source is arranged along a spray chain which is kept at a distance from the floor and lateral wall areas by one or more spacers.

10. The device of claim 9, wherein the at least one source of electromagnetic radiation is a source of UV light.

11. The device of claim 9, wherein each of the one or more spacers has a free end, and additionally includes one or more slide shoes or rollers at their free end regions.

12. The device of claim 9, wherein each of the one or more spacers is adjustable in length.

13. The device of claim 9, wherein the device includes at least three spacers that point in different circumferential directions.

14. The device of claim 9, including a spray head configured to dispense disinfectant, where the disinfectant is selected from among halogen- and/or oxygen- and/or nitrogen-containing chemicals and/or alcohols.

15. The device of claim 9, wherein the at least one source of electromagnetic radiation includes one or more UVC or UV LED lamps.

16. The device of claim 15, wherein the source of electromagnetic radiation is configured to emit light in the wavelength range of between 200-300 nm.

17. The device of claim 9, further comprising at least one nozzle for dispensing gaseous CO₂ is fitted on the spray chain.

18. A method of disinfecting a pipeline, container, or structure having a floor and lateral walls, the method comprising:
urging a spray chain through the pipeline, container, or structure where the spray chain is equipped with at least one spray head configured to dispense disinfectant and/or at least one source of electromagnetic radiation arranged along the spray chain, wherein the spray chain is kept at a distance from the floor and lateral wall areas by one or more spacers;
dispensing disinfectant and/or emitting electromagnetic radiation from the at least one spray head and/or at least one source of electromagnetic radiation.

19. The method of claim 18, further comprising dispensing gaseous CO₂ from at least one nozzle fitted on the spray chain.
